(19) United States
(12) Patent Application Publication
(10) Pub. No.: US 2020/0023089 A1
(43) Pub. Date: Jan. 23, 2020

(54) PORTABLE CLEANING SYSTEM

(71) Applicant: Kraft Foods Group Brands LLC, Chicago, IL (US)

(72) Inventor: James Richard Hosford, Murfreesboro, TN (US)

(21) Appl. No.: 16/040,073

(22) Filed: Jul. 19, 2018

(51) Int. Cl. A61L 2/07; A61L 2/26; B65H 75/40; B65H 75/44

(52) U.S. Cl. CPC A61L 2/07; A61L 2/26; B65H 75/403; B65H 2402/41; A61L 2202/16; B65H 2301/5115; B65H 2701/3911; B65H 75/4492

(57) ABSTRACT

Described herein is a portable cleaning system comprising a reel around which one or more elongated flexible machine components such as roller chains may be wound to withdraw them from a machine, and means to clean and sanitize the components while they are supported on the reel. The reel may be rotatably supported on a cart. The apparatus further includes an enclosure for the reel, and means for introducing a fluid such as steam into the enclosure to clean and sanitize the components.

### TECHNICAL FIELD

This application relates generally to cleaning systems, and specifically to portable cleaning systems that may be suitable for use in food plants and other controlled environments.

### BACKGROUND

Methods for sanitizing equipment used in controlled production environments, e.g., in food, pharmaceutical, and biopharmaceutical plants, include cleaning in place (CIP), cleaning out of place (COP), immersion cleaning, and combinations thereof. Because cleaning time can reduce production time, and because frequent cleaning may be required, quickness and efficiency in cleaning operations can be highly valued.

One particular challenge involves cleaning of equipment components that have close-fitting parts between which matter may become trapped. Cleaning and sanitizing roller chains can be especially challenging. Removing chains from machines, transporting them to a cleaning location, and thereafter replacing them in the machine while avoiding kinking or tangling or portions of the chains can be problematic.

CIP methods can sanitize some equipment without moving the equipment from its operating area. Hoses and/or piping may be used to provide water, steam and/or other fluids to difficult-to-reach areas. However, efficient cleaning of certain parts such as chains in form-fill-seal machines using CIP techniques can be difficult or impossible due to the lengths of the chains and other factors. After a CIP operation, removal of residual cleaning fluid from an operating area can take additional time.

COP methods remove equipment from an operating area to a separate cleaning area. Although COP methods may be preferred for certain equipment and components, COP methods can be inefficient and time-consuming due to the time required for disassembly, movement of the equipment and components to be cleaned, and reassembly.

Immersion methods submerge equipment into cleaning solutions that can penetrate into difficult to reach areas, and provide extended periods of contact between cleaning solutions and surfaces to be cleaned. While these methods can be very effective, immersion cleaning has the disadvantage of requiring a vessel large enough to hold the equipment to be cleaned, and a large enough volume of cleaning solution to enable immersion. In addition, lifting and lowering articles to be cleaned can present logistical issues, e.g., in positioning the articles at an elevation above the fluid surface, then lowering them into the fluid, then lifting them from the fluid and returning them to an operating location. Also, immersion may involve application of relatively low mechanical forces to surface being cleaned, as compared with, e.g., scrubbing or high-pressure spraying.

### SUMMARY

Described herein is a portable cleaning system or apparatus comprising a reel around which one or more elongated flexible machine components such as roller chains may be wound to withdraw them from a machine, and means to clean and sanitize the components while they are supported on the reel. The reel may be rotatably supported on a cart. The apparatus further includes an enclosure for the reel.

The reel includes a conduit for receiving a fluid. The conduit may be a hollow shaft or axle which supports the reel for rotation on the cart, with the conduit being at the center of rotation. The conduit has a plurality of openings spaced along its peripheral surface to distribute fluid radially outward from the conduit within the enclosure to clean and sanitize the elongated flexible machine components within the enclosure. In some embodiments, the fluid is a heated fluid such as steam, and the sanitation process comprises heating the entire interior of the enclosure to raise the temperature of the components.

To provide fluid to the conduit, the portable cleaning apparatus may include a fluid source such as a flexible hose or pipe that provides steam to the conduit, and a quick-disconnect fitting or other connector to join the fluid source to the conduit. The fluid source may be coupled to the reel at one end, and a handle may be coupled to the reel at an opposite end to facilitate manual rotation of the reel about its axis.

The enclosure may comprise a removable flexible film wrapped around the elongated flexible machine component and temporarily secured in place to facilitate maintenance of an elevated temperature and concentration of steam within the enclosure.

In some embodiments, the reel comprises first and second end walls to define a region of the reel on which elongated machine components may be wound, with the elongated machine components and the plurality of openings in the conduit being disposed between the end walls. The portable cleaning apparatus may further include a divider or an intermediate divider wall between the first and second end walls to divide the region into two smaller areas, each of which is capable of accommodating a chain from a form-fill-seal machine wound on the reel. The intermediate divider wall may have one or more openings therethrough to permit fluid to flow therethrough, thereby preventing unacceptably high variations in temperature on opposite sides of the divider.

The reel may further comprise a plurality of struts extending parallel to the axis of the reel to (1) increase the strength and stiffness of the reel, (2) provide a structure having increased radial dimensions to facilitate winding the chains on the reel without requiring an excessive number of rotations, and (3) maintain spacing between elongated flexible machine components and the openings in the conduit to prevent elongated flexible machine components from undesirably obstructing flow of fluid through the openings.

A plurality of devices for measuring temperatures may be positioned at spaced locations in the enclosure, and a device for recording the measured temperatures at a plurality of points in time may be coupled to the devices for measuring temperatures. The portable cleaning apparatus may be capable of maintaining and verifying maintenance of an elevated temperature within the enclosure for an extended period of time, e.g., a temperature of 140° F. to 190° F. or a temperature of 160° F. to 180° F. within the enclosure for a period of 20, 30 or 40 minutes.

There is disclosed a method of cleaning and sanitizing a chain comprising positioning a portable cleaning apparatus adjacent a machine that includes at least one chain, the portable cleaning apparatus comprising a cart and a reel rotatably supported on the cart; decreasing chain tension, e.g., by adjusting a tensioner, and thereby loosening the chain in the machine; disconnecting a master link or otherwise disconnecting two adjacent links of the chain to change the configuration of the chain from a closed loop to a configuration in which the chain has a leading end and a trailing end; withdrawing the leading end of the chain from the machine and attaching it to the reel; rotating the reel to withdraw the chain entirely from the machine and wind the chain on the reel; securing the trailing end of the chain; providing an enclosure about the chain on the reel; introducing a heated fluid into the enclosure to clean and sanitize the chain; and replacing the chain on the machine by threading the chain into the machine and unwinding the chain from the reel. The method may enable cleaning and sanitizing of the chain without permitting the chain to contact the floor.

Some or all of the above steps may be performed on two or more chains simultaneously, with the reel including one or more dividers to prevent the respective chains from engaging each other, thereby avoiding possible entanglement. The divider may have one or more openings therein to permit fluid to flow therethrough during cleaning and sanitizing operations.

In some embodiments, replacing the chains on the machine may comprise using a machine drive component to apply tension to the chains and pull the chains from the reel into the machine, and thereby rotating the reel to unwind the chains from the reel.

End walls of the reel may form part of the enclosure, and may constrain the chains against axial displacement to keep them on the reel. Providing an enclosure may comprise wrapping a flexible film about the chains between the end walls, or wrapping a flexible film about the entire reel, or using another flexible or rigid material to enclose the chains and/or the entire reel. Limited escape of steam from the enclosure may occur through openings or gaps in the enclosure material, and/or through gaps between the enclosure material and the reel, or through one or more other openings.

In the above method, providing heated fluid may comprise providing pressurized steam to one end of a centrally located conduit on the reel, and forcing steam radially outward through a plurality of openings in the conduit, wherein the centrally located conduit may function as an axis of rotation for the reel, and wherein a plurality of struts disposed radially outward of the centrally located conduit may engage the end walls and the divider to (1) increase the strength and stiffness of the reel, (2) provide a structure having increased radial dimensions to facilitate winding the chains on the reel without requiring an excessive number of rotations, and (3) maintain spacing between the chains and the openings in the conduit to prevent the chains from undesirably obstructing flow of heated fluid through the openings in the conduit.

The method may include providing at least one temperature sensor in the enclosure, measuring the temperatures at multiple points in the enclosure, and recording the temperatures at multiple points in time. Effecting flow of steam and/or other heated fluids into the enclosure may result in the temperature being measured at multiple points in the enclosure as being between 160° F. and 180° F. for at least 30 minutes, with such measurements being recorded using a chart recorder other device to document the temperature measurements and the timing of the measurements.

After replacement of the chains in the machine, the portable cleaning apparatus may be removed from the position adjacent the machine.

### BRIEF DESCRIPTION OF THE DRAWINGS

FIG. 1. illustrates a portable cleaning system.

FIG. 2 is a schematic isometric illustration of a reel for use in a portable cleaning system similar to that of FIG. 1.

FIG. 3 is a schematic front elevation of the reel.

FIG. 4 is a schematic side elevation of the reel, taken from the left-hand side of FIG. 2.

FIG. 5 is a schematic side elevation of a reel divider

FIG. 6 is a schematic side elevation of the reel, taken from the right-hand side of FIG. 2.

FIG. 7 illustrates the reel of FIG. 2 with a pair of chains and an enclosure thereon.

FIG. 8 illustrates use of a portable cleaning system to remove chains from a machine.

### DETAILED DESCRIPTION

Described herein in is a portable cleaning system for machine parts. The term “machine” is used broadly herein to encompass any equipment or device. In some embodiments, the machine parts to be cleaned may be used in manufacturing or processing plants related to food, pharmaceutical, biopharmaceutical, or other industries. In some embodiments, the machine parts may be chains that are used in form fill seal machines, including, e.g., Multivac, Power Pak, Evolution, Hamba, and Waltrop Pin/Chain Line machines, other chain-driven line machines and other equipment.

The portable cleaning system may provide an efficient way to remove elongated flexible components such as chains from machines, and may further provide an enclosure containing a localized region of increased temperature to sanitize the components. In some applications, the system may be used in a food plant or other environment in which the air is at room temperature or cooler than room temperature. In some applications, the system may be used with surrounding air at temperatures of, e.g., 35° F. to 65° F., 45° F. to 55° F., or at higher or lower temperatures.

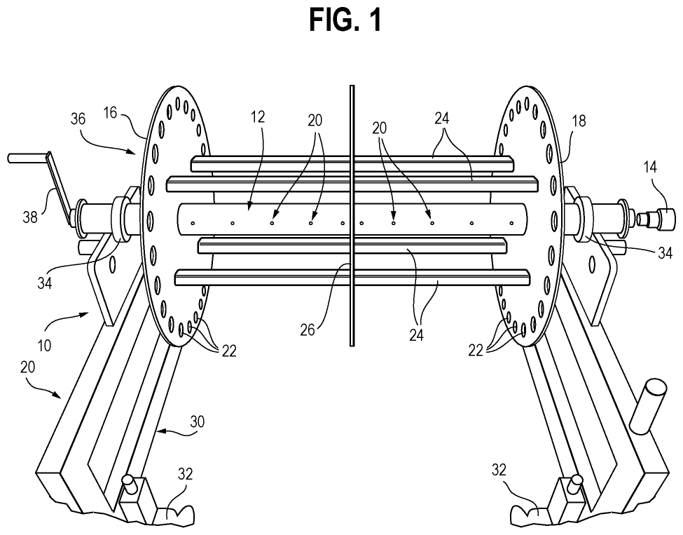

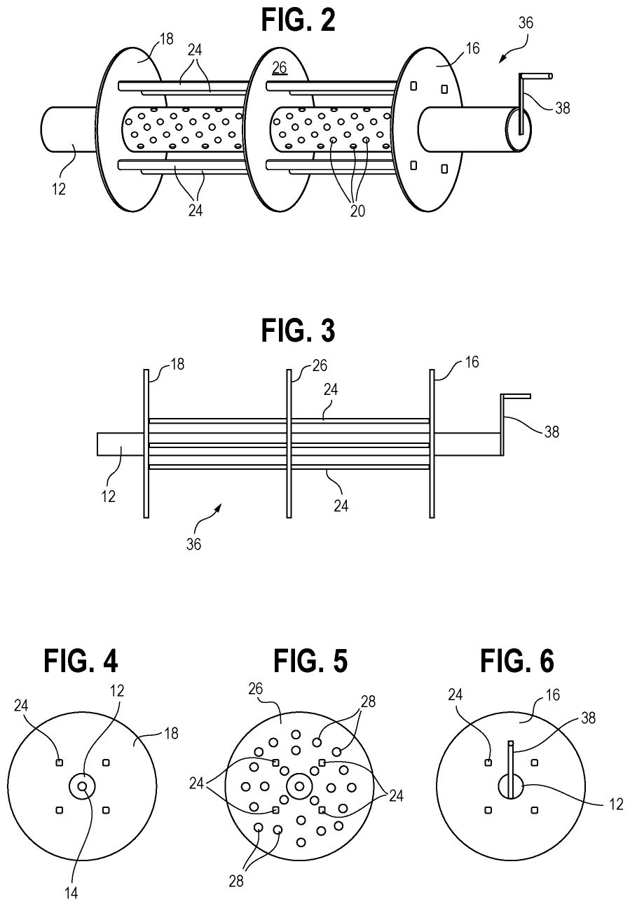

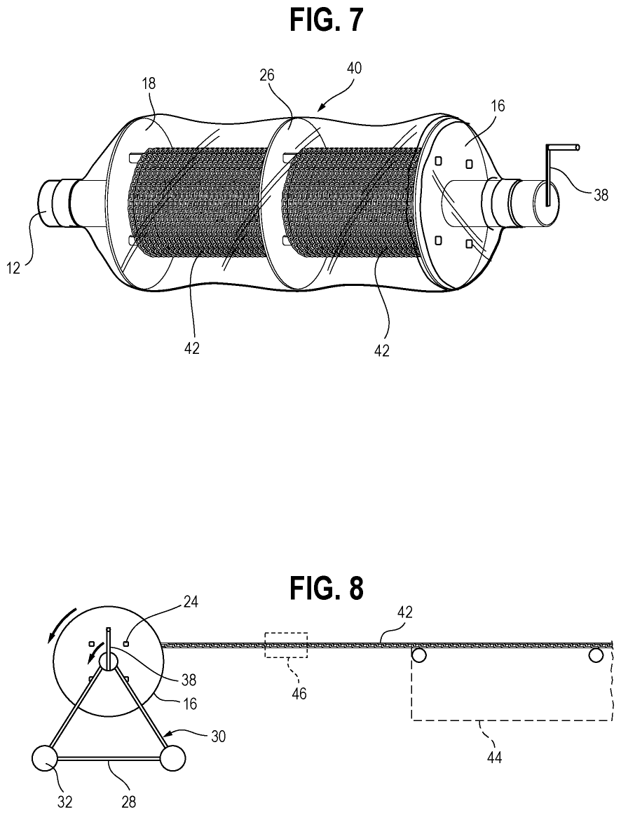

FIGS. 1-8 illustrate a portable cleaning system or apparatus 10 comprising a reel 36 around which one or more elongated flexible machine components 42 such as roller chains may be wound to withdraw them from a machine 44, and means to clean and sanitize the components 42 while they are supported on the reel 36. The reel 36 may be rotatably supported on semicircular seats 34 on a cart 30 having wheel assemblies 32 to facilitate positioning and transport. The apparatus 10 further includes an enclosure 40 for the reel.

The reel 36 includes a conduit 12 for receiving a fluid. The conduit 12 may be a hollow shaft or axle which supports the reel 36 for rotation on the cart 30, with the conduit 12 being at the center of rotation. The conduit 12 has a plurality of openings 20 spaced along its peripheral surface to distribute fluid radially outward from the conduit within the enclosure 40 to clean and sanitize the elongated flexible machine components 42 within the enclosure 40. In some embodiments, the fluid is a heated fluid such as steam, and the sanitation process comprises heating the entire interior of the enclosure 40 to raise the temperature of the components 42.

To provide fluid to the conduit 12, the portable cleaning apparatus 10 may include a fluid source such as a flexible hose or pipe that provides steam to the conduit 12, and a quick-disconnect fitting or other connector 14 to couple the fluid source to the conduit 12. The fluid source may be coupled to the reel 36 at one end, and a handle 38 may be coupled to the reel 36 at an opposite end to facilitate manual rotation of the reel 36 about its axis. The connector 14 may have a central opening for passage of fluid with a diameter of ¼ to 2 in., ½ to 1½ in., about 1 in., or another dimension.

The enclosure 40 may comprise a removable flexible film wrapped around the elongated flexible machine component 42 and temporarily secured in place to facilitate maintenance of an elevated temperature and concentration of steam within the enclosure 40. In some embodiments, the enclosure 40 may cover, enclose, envelop, or encase the flexible machine component 42 without necessarily being wrapped around the component 42.

In some embodiments, the reel 36 comprises first and second end walls 16 and 18 to define a region of the reel 36 therebetween on which elongated machine components 42 may be wound, with the elongated machine components 42 and the plurality of openings 20 in the conduit 12 being disposed between the end walls 16, 18. The portable cleaning apparatus 10 may further include an intermediate divider wall 26 between the first and second end walls 16, 18 to divide the region into two smaller areas, each of which is capable of accommodating a chain 42 from a form-fill-seal machine wound on the reel 36. The intermediate divider wall 26 may have one or more openings 28 therethrough to permit fluid to flow therethrough, thereby preventing unacceptably high variations in temperature on opposite sides of the intermediate divider wall 26.

The reel 36 may further comprise a plurality of struts 24 extending parallel to the axis of the reel 36 to (1) increase the strength and stiffness of the reel, (2) provide a structure having increased radial dimensions to facilitate winding the chains 42 on the reel 36 without requiring an excessive number of rotations, and (3) maintain spacing between elongated flexible machine components 42 and the openings 20 in the conduit 12 to prevent elongated flexible machine components 42 from undesirably obstructing flow of fluid through the openings 20.

A plurality of devices for measuring temperatures may be positioned at spaced locations in the enclosure, and a device for recording the measured temperatures at a plurality of points in time may be coupled to the devices for measuring temperatures. The portable cleaning apparatus 10 may be capable of maintaining and verifying maintenance of an elevated temperature within the enclosure 40 for an extended period of time, e.g., a temperature of 160° F. to 180° F. within the enclosure for a period of 30 minutes.

A method of cleaning and sanitizing a chain 42 may comprise positioning a portable cleaning apparatus 10 adjacent a machine 44 that includes at least one chain 42, the portable cleaning apparatus 10 comprising a cart 30 and a reel 36 rotatably supported on the cart 30; decreasing chain tension in the machine 44, e.g., by adjusting a tensioner, and thereby loosening the chain 42 in the machine; disconnecting a master link or otherwise disconnecting two adjacent links of the chain 42 to change the configuration of the chain 42 from a closed loop to a configuration in which the chain 42 has a leading end and a trailing end; withdrawing the leading end of the chain 42 from the machine 44 and attaching it to the reel 36; rotating the reel 36 to withdraw the chain 42 entirely from the machine 44 and wind the chain 42 on the reel 36 as shown in FIG. 8; securing the trailing end of the chain 42 to the reel 36 or to a portion of the chain 42; providing an enclosure 40 about the chain 42 on the reel 36; introducing a heated fluid into the enclosure 40 to clean and sanitize the chain 42; and replacing the chain 42 on the machine 44 by threading the chain 42 into the machine 44 and unwinding the chain 42 from the reel 36. The method may enable efficient cleaning and sanitizing of the chain 42 without permitting the chain 42 to contact the floor.

Attaching the leading end to the reel 36 may comprise attaching the leading end to the conduit, a strut, an end wall or the divider with a wire tie or other fastening device.

Some or all of the above steps may be performed on two or more chains simultaneously, with the reel 36 including one or more intermediate divider walls 26 to prevent the respective chains from engaging each other, thereby avoiding possible entanglement. The intermediate divider wall 26 may comprise one or more vertical discs oriented perpendicularly to the axis of rotation of the reel and having one or more openings therein to permit fluid to flow therethrough during cleaning and sanitizing operations.

In some embodiments, replacing the chains on the machine may comprise using a machine drive component to apply tension to the chains and pull the chains from the reel into the machine, and thereby rotating the reel to unwind the chains from the reel.

Reel end walls 16 and 18 may form part of the enclosure, and may constrain the chains against axial displacement to keep them on the reel 36. Providing an enclosure 40 may comprise wrapping a flexible film about the chains 42 between the end walls 16 and 18, or wrapping a flexible film about the entire reel 36, or using another flexible or rigid material to enclose the chains and/or the entire reel 36. Limited escape of steam from the enclosure may occur through openings or gaps in the enclosure material, and/or through gaps between the enclosure 40 and the reel 36.

Providing heated fluid may comprise providing pressurized steam to one end of the centrally located conduit 12 on the reel 36, and forcing steam radially outward through a plurality of openings 20 in the conduit 12, wherein the centrally located conduit 12 may function as an axis of rotation for the reel 36, and wherein a plurality of struts 24 disposed radially outward of the centrally located conduit may engage the end walls 16, 18 and the intermediate divider wall 26 to (1) increase the strength and stiffness of the reel, (2) provide a structure having increased radial dimensions to facilitate winding the chains on the reel without requiring an excessive number of rotations, and (3) maintain spacing between the chains and the openings in the conduit to prevent the chains from undesirably obstructing flow of heated fluid through the openings in the conduit. The struts may be made of 1 in. square tubing.

The method may include providing at least one temperature sensor in the enclosure, measuring the temperatures at multiple points in the enclosure, and recording the temperatures at multiple points in time. Effecting flow of steam and/or other heated fluids into the enclosure may result in the temperature being measured at multiple points in the enclosure as being between 160° F. and 180° F. for at least 30 minutes, with such measurements being recorded using a chart recorder other device to document the temperature measurements and the timing of the measurements.

After replacement of the chains in the machine, the portable cleaning apparatus may be removed from the position adjacent the machine.

In some embodiments, the conduit 12 may comprise a pipe, tube, or other hollow elongated member having an external diameter of, e.g., 1 to 4 in., 2 to 3 in. or about 2.5 in. The conduit 12 may be positioned substantially horizontally between first and second substantially vertical end walls 16 and 18. The conduit may have a generally cylindrical peripheral surface with the openings 20 formed therein to enable dispersion of fluid from the interior to the exterior of the conduit 12. In some embodiments, the openings 20 may be in a pattern, such as a line pattern, helical pattern, zigzag pattern, sinusoidal pattern, circular pattern, or a square pattern. The openings 20 may be of the same or different sizes, and may have diameters of, e.g., from 1/64 in. to 1 in., 1/64 in. to ¼ in., or about ⅛ in. In some embodiments, the openings 20 may be spaced 1 in. to 5 in. apart, 2 in. to 4 in. apart, or about 3 in. apart. In various embodiments, the openings 20 may be spaced around the entirety of the conduit, or around only portions thereof.

The first and second end walls 16, 18 may have openings 22 that can provide connection points for attachment of chains of other components to be cleaned.

In some embodiments, the intermediate divider wall 26 may not have any openings. In some embodiments, the openings 28 in the intermediate divider wall 26 may be sized from about ¼ in. to 1¾ in., ½ in. to 1½ in. or about 1 in.

In some embodiments, the end walls 16, 18 may have a diameter of about 15 to 25 in., 18 to 22 in., or about 20 in. In some embodiments, the end walls 16, 18 may be larger or smaller than these ranges to facilitate cleaning of larger or smaller chains or other components. In some embodiments, the struts 24 may positioned be 3 to 9 in., 5 to 7 in. or about 6 in. radially inward from the outer edges of the end walls, and may be at a radius of about 1 to 7 in., 3 to 5 in., or about 4 in. from the axis of the reel. In some embodiments, the struts 24 may be closer to or farther from the axis and/or the outer edges of the end walls 16, 18. The conduit, end walls, and struts may be made of stainless steel with a gauge suitable for the weight of the chains or other components that they are to be used with. In some embodiments, 10-gauge stainless steel may be employed for the end walls and intermediate wall(s). The conduit, end walls, and intermediate wall may alternatively be made of other suitable materials

In some embodiments, alternatives to the illustrated handle 38 may include a wheel or a ratchet. In some embodiments, a motor or other drive mechanism may be provided to rotate the reel. In some embodiments, handle 38 may be attached by a weld or otherwise coupled to the conduit 12 or an end wall 16, 18. In some embodiments, handle 38 may have a width of 2 to 6 in., 3 to 5 in., or about 4 in. In various embodiments, handle 38 may have a length of 6 to 14 in, 8 to 12 in., or about 12 in. In some embodiments, the end of the conduit 12 where handle 38 is attached is sealed by an end cap. In some embodiments, as one or more chains 42 are pulled from a machine 44 by the reel 36, a guide 46 may manually help guide the chain onto the reel so that the chain does not kink and is evenly distributed on the reel 36. In some embodiments, the guide 46 may be a person. In other embodiments, the guide 46 may be a mechanical device or instrument. In some cases, two or more operators may work together to carry out the cleaning and sanitation process.

A leading portion of the chain may be temporarily attached to the reel 36, or specifically to the conduit 12, a strut 24, an end wall 16, 18 or the intermediate divider wall 26 with a wire tie or other fastening device.

Multiple machine components 42 such as chains may be attached and loaded onto the reel 36 concurrently or consecutively, which may affect the number of individuals needed to assist with the operation of the portable cleaning system 10. While one individual rotates the handle 38, one or more other individuals may guide 46 the chains 42 to help ensure that they roll evenly onto the reel 36 and that the chains 42 do not kink or tangle as they are removed from the machine 44. After the chains 42 are completely removed from the machine 44 and loaded onto the reel 36, the trailing end of each chain may be secured to the respective chain or to the reel 36 with a fastening device such as a wire tie, a cord, or other suitable device. In some embodiments, to provide an enclosure 40 or containment structure about the chains 42, the reel 36 may then be wrapped in a plastic film, which may be secured with duct tape or other adhesive tape, or other means such as snaps, zippers, buttons, hook and loop fasteners, or elastic or inelastic materials such as bands, straps or cords. In some embodiments, other flexible or rigid material may be used to enclose the chains during cleaning, and to concentrate steam to enable the chains to be heated to a desired temperature for a desired length of time without requiring excessive amounts of steam to be introduced into the enclosure, and without releasing excessive amounts of steam to the surrounding environment.

Probes, thermometers, and/or other means to measure temperature may be inserted through the plastic or otherwise provided within the enclosure to read the temperature of the steam, air and/or other fluid in proximity to the chains, thus enabling the approximate temperature of the chains themselves to be determined. Probes may be secured with a tape such as duct tape or other means to reduce or prevent steam leakage around the probes. The devices for measuring temperature may be connected to a chart recorder, which allows for a written recording of the results of the temperature probes at various specific points in time.

In some embodiments, the steam input results in temperatures in the enclosure, i.e., at or near the chains, being within a desired temperature range, e.g., 140 to 200 F, 150 to 190 F, or 160 to 180 F, for 20 to 40 minutes, 25 to 35 minutes, or about 30 minutes. The chart recorder may document the temperature outputs for the entire length of time.

The cart 30 may be a conventional cart of a type used to move reels of packaging film, or may another type of cart.

In various approaches, the chains may be cleaned out-of-place, and the parts of the machine that are not removed (e.g., the chain guides) may be cleaned in place. In some embodiments, the cleaning-in-place of the chain guides may involve the use of one or more devices such as hoses, foamers, buckets, brushes, wipes, squeegees, spray bars, steam machines, scrapers, vacuums, air hoses, and air guns.

In some approaches, employment of the portable cleaning system may be part of comprehensive cleaning regime that may include disassembly of machinery or equipment; dry cleaning; initial rinsing; soaping and scouring; post-rinsing and inspection of machinery and equipment for remaining debris; removal of standing water; cleaning of parts that are inaccessible when a machine is assembled; assembly of machinery or equipment; inspection for remaining chemicals, tools, and cleaning supplies; and disinfection and sanitization.

Those skilled in the art will recognize that a wide variety of modifications, alterations, and combinations may be made with respect to the above described embodiments without departing from the scope of the invention, and that such modifications, alterations, and combinations are to be viewed as being within the ambit of the inventive concept.

What is claimed is:

1. A portable cleaning apparatus comprising:
a reel around which a pair of elongated flexible machine components may be wound; and
an enclosure for the reel;
the reel comprising a conduit for receiving a fluid;
the conduit having a plurality of openings therein to distribute the fluid within the enclosure to clean and sanitize elongated flexible machine components within the enclosure.

2. The portable cleaning apparatus of claim 1 further comprising a source of steam coupled to the conduit.

3. The portable cleaning apparatus of claim 2 wherein the enclosure comprises a removable flexible film wrapped around the elongated flexible machine component and temporarily secured in place to facilitate maintenance of an elevated temperature and concentration of steam within the enclosure.

4. The portable cleaning apparatus of claim 2 wherein the reel comprises first and second end walls to define a region of the reel on which elongated machine components may be wound, with the plurality of openings in the conduit being disposed between the end walls.

5. The portable cleaning apparatus of claim 4, wherein the reel further comprises an intermediate divider wall between the first and second end walls to divide the region into two smaller areas, each smaller area being capable of accommodating a chain from a form-fill-seal machine wound on the reel.

6. The portable cleaning apparatus of claim 5 further comprising a plurality of struts parallel to the axis of the reel to increase the strength and stiffness of the reel, provide a structure having increased radial dimensions to facilitate winding the chains on the reel without requiring an excessive number of rotations, and maintain spacing between elongated flexible machine components and the openings in the conduit to prevent elongated flexible machine components from undesirably obstructing flow of fluid through the openings.

7. The portable cleaning apparatus of claim 6 wherein the intermediate divider wall has at least one opening to permit cleaning fluid to flow therethrough.

8. The portable cleaning apparatus of claim 7 wherein the source of steam comprises a flexible hose coupled to the reel at one end, and wherein the portable cleaning apparatus further comprises a handle coupled to the reel at an opposite end to facilitate manual rotation of the reel about its axis.

9. The portable cleaning apparatus of claim 1 further comprising a device for measuring temperatures at multiple locations in the enclosure, and a device for recording the measured temperatures at a plurality of points in time.

10. The portable cleaning apparatus of claim 1 wherein the portable cleaning apparatus is capable of maintaining a temperature of 160 to 180 F within the enclosure for 30 minutes.

11. The portable cleaning apparatus of claim 1 further comprising a cart, the reel being supported for rotation on the cart, the cart including a plurality of wheel assemblies to facilitate transport of the portable cleaning apparatus.

12. A method of cleaning and sanitizing a chain used in a machine, comprising:
a. positioning a portable cleaning apparatus adjacent a machine that includes at least one chain, the portable cleaning apparatus comprising a cart and a reel rotatably supported on the cart;
b. temporarily and removably attaching the at least one chain to the reel;
c. rotating the reel to withdraw the at least one chain from the machine and wind the at least one chain on the reel;
d. providing an enclosure about the at least one chain on the reel;
e. introducing a heated fluid into the enclosure to clean and sanitize the chain;
f. replacing the chain on the machine; and
g. removing the portable cleaning apparatus from the position adjacent the machine.

13. The method of claim 12 wherein the at least one chain comprises a pair of chains, the reel includes a divider, the chains are attached to the reel on opposite sides of the divider and are prevented from engaging each other by the divider to avoid entanglement, the divider has at least one opening therein, and the heated fluid flows through the at least one opening during cleaning.

14. The method of claim 13 wherein replacing the chains on the machine comprises using a machine drive component to apply tension to the chain and pull the chains from the reel into the machine, and thereby rotating the reel to unwind the chains from the reel.

15. The method of claim 14 further comprising using end walls of the reel to form part of the enclosure and to constrain the chains against axial displacement.

16. The method of claim 15 wherein providing an enclosure comprises providing a flexible film.

17. The method of claim 16 wherein introducing a heated fluid comprises providing pressurized steam to one end of a centrally located conduit on the reel and forcing steam radially outward through a plurality of openings in the conduit, and wherein the centrally located conduit functions as an axis of rotation for the reel, and wherein a plurality of struts disposed radially outward of the centrally located conduit engage the end walls and the divider to (1) increase the strength and stiffness of the reel, (2) provide a structure having increased radial dimensions to facilitate winding the chains on the reel, and (3) maintain spacing between the chains and the openings in the conduit to facilitate flow of heated fluid through the openings.

18. The method of claim 17 wherein introducing heated fluid into the enclosure comprises introducing steam into the enclosure, and wherein the method further comprises providing at least one temperature sensor in the enclosure.

19. The method of claim 18 wherein introducing steam into the enclosure results in a temperature in the enclosure being at least 160° F. for at least 30 minutes, and wherein the temperature in the enclosure is recorded at multiple points in time during cleaning and sanitation.
